(19) United States
(12) Patent Application Publication
(10) Pub. No.: US 2009/0209654 A1
(43) Pub. Date: Aug. 20, 2009

(54) Prenylflavonoid Formulations

(76) Inventor: Eric Kuhrts, Bodega, CA (US)

Correspondence Address: Kenneth E. Jenkins;Townsend and Townsend and Crew LLP, Two Embarcadero Center, Eighth Floor, San Francisco, CA, 94111-3834, US

(21) Appl. No.: 11/989,739

(22) PCT Filed: Jul. 31, 2006
(86) PCT No.: PCT/US2006/029962

§ 371 Date: Dec. 19, 2008

Related U.S. Application Data
(60) Provisional application No. 60/703,677, filed on Jul. 29, 2005.

(51) Int. Cl. A61K 31/12; C07C 45/80; A61P 3/10

(52) U.S. Cl. 514/685; 568/309

(57) ABSTRACT

Methods and formulations for increasing the water solubility and/or bioavailability of prenylfiavonoids are disclosed. The formulations may be employed to treat a disease states, including cancer.

### BACKGROUND OF THE INVENTION

Flavonoids are abundant throughout nature and exert a broad range of biological activities in plants and animals. There are now considered to be over 4,000 flavonoids existent in nature. Some of the biological activities of flavonoids include; anti-inflammatory, antiviral, antifungal, antibacterial, estrogenic, anti-oxidant, antiallargenic, anticarcinogenic, and antiproliferative medicinal properties.

Hops (Humulus lupulis L.) has been used for centuries as a bittering agent in the brewing of beer. Hops contains alpha acids such as humulone, co-humuone, ad-humulone, and beta acids such as lupulone and co-lupulone. Hops also contains many flavonoids, such as xanthohumol, isoxanthohumol, desmethylxanthohumol, 8-prenylnaringenin, and 6-prenylnaringenin. Xanthohumol is a yellow-orange substance with a melting point of 172 degrees C. A typical ethanol extract of hops yields about 3 mg./g (3%) of xanthohumol out of a total flavonoid content of 3.46 mg./g. Dried hop contains about 0.2 to 1.0% by weight xanthohumol.

Xanthohumol and other hop prenylflavonoids have been identified as cancer chemopreventive agents through their interfering action with a variety of cellular mechanisms at low micromolar concentrations such as (1) inhibition of metabolic activation of procarcinogens, (2) induction of carcinogen-detoxifying enzymes, and (3) inhibition of tumor growth by inhibiting inflammatory signals and angiogenesis. Stevens, et al., Phytochemistry 65: 1317-1330 (2004). See also Stevens, et al, Chemistry and Biology of Hops Flavonoids; and Stevens, J. Am. Soc. Brew. Chem. 56(4): 136-145 (1998). Antiproliferative and cytotoxic effects of xanthohumol and five other prenylated hop flavonoids were tested in breast cancer (MCF-7), colon cancer (HT-29), and ovarian cancer (A-2780) cells in vitro. Miranda, et al. Drug Metab. Dispos. 28: 1297-1302 (1999). Xanthohumol inhibited the proliferation of MCF-7 and A-2780 cells in a dose-dependent manner with IC₅₀ values of 13 and 0.52 M, respectively, after two days of treatment. Gerhauser et al. showed that xanthohumol can be an effective anti-inflammatory agent by inhibition of endogenous prostaglandin synthesis through inhibition of cyclooxygenase (constitutive COX-1 and inducible COX-2) enzymes with IC50 values of 17 and 42 μM, respectively. Gerhauser et al., Mol. Cancer Ther. 1: 959-969 (2002). Xanthohumol, isoxanthohumol, 8-prenylnaringenin, and nine other prenylflavonoids from hops were shown to strongly inhibit the cDNA-expressed human cytochrome P450 enzymes, Cyp1A1, Cyp1B1, and Cyp1A2 (Henderson et al., Xenobiotica 30: 235-251 (2000). The effect of 8-prenylnaringenin on angiogenesis was studied by Pepper et al., who demonstrated that 8-prenylnaringenin inhibits angiogenesis in an in vitro model in which endothelial cells can be induced to invade a three-dimensional collagen gel and form capillary-like tubes. Pepper et al., J. Cell Physiol. 199: 98-10 (2004).

Ethanol may be used to extract higher levels of the prenylflavonoids from hops. The typical prenylflavonoid content of an ethanol extract of hops includes xanthohumol (3 mg/g), desmethylxanthohumol (0.34 mg./g), isoxanthohumol (0.052 mg/g), 6-prenylnaringenin (0.061 mg/g), and 8-prenylnaringenin 0.015 (mg/g). Supercritical carbon dioxide extractions tend to contain much lower levels, or non-existent levels of prenylflavonoids. In fact, these compounds are almost non-existent in standard CO₂ extracts because the prenylflavonoids are virtually insolvent on carbon dioxide.

In order for any therapeutic molecular substance to be transported through the membranes of the human body, the molecule must be dissolvable in the aqueous phase of the intestinal fluid. Without dissolution, the drug would pass through the GI-tract as would brick-dust. Prenylflavonoids such as xanthohumol are virtually insoluble in water, and animal pharmacokinetic studies of oral doses have demonstrated very low bioavailability.

Due to the many desirable properties of prenylflavonoids, it would be advantageous to have a more water soluble formulation and/or enhanced bioavailability of a prenylflavanoid for dosing in-vivo. The present invention solves these and other problems in the art.

### BRIEF DESCRIPTION OF THE DRAWINGS

FIG. 1 illustrates cholesterol synthesis inhibition by xanthohumol in a dose-responsive manner in HepG2 Cells as % of Control Activity with concentration in μM.

### BRIEF SUMMARY OF THE INVENTION

In one aspect, the present invention provides a water-soluble formulation including a prenylflavonoid or prenylflavonoid metabolite, and a non-ionic surfactant.

In another aspect, the present invention provides a method of treating cancer, obesity, diabetes, cardiovascular disease, dyslipidaemia, age-related macular degeneration (e.g. vision loss associated with age-related macular degeneration), high cholesterol, or retinopathy (e.g. diabetic retinopathy) in subject in need of such treatment. The method includes administering to the subject an effective amount of the water soluble formulation of the present invention.

In another aspect, the present invention provides a method of treating a VEGF-mediated disease state in a subject in need of such treatment. The method includes administering to the subject an effective amount of the water soluble formulation of the present invention.

In another aspect, the present invention provides a method of treating a DGAT-mediated disease state in a subject in need of such treatment. The method includes administering to the subject an effective amount of the water soluble formulation of the present invention.

In another aspect, the present invention provides a method of treating a ACAT-mediated disease state in a subject in need of such treatment. The method includes administering to the subject an effective amount of the water soluble formulation of the present invention.

In another aspect, the present invention provides a method for enhancing the bioavailability of a prenylflavonoid or prenylflavonoid metabolite in a subject. The method includes combining the prenylflavonoid or prenylflavonoid metabolite, and a non-ionic surfactant to form a surfactant-prenylflavonoid mixture. The surfactant-prenylflavonoid mixture is administered to the subject thereby enhancing the bioavailability of the prenylflavonoid or prenylflavonoid metabolite.

In another aspect, the present invention provides a method of dissolving a prenylflavonoid in water. The method includes combining a prenylflavonoid with a non-ionic surfactant to form a surfactant-prenylflavonoid mixture. The surfactant-prenylflavonoid mixture is combined with water thereby dissolving the prenylflavonoid in water.

### DETAILED DESCRIPTION OF THE INVENTION

### I. Definitions

The abbreviations used herein have their conventional meaning within the chemical and biological arts.

The term “pharmaceutically acceptable salts” is meant to include salts of the active compounds which are prepared with relatively nontoxic acids or bases, depending on the particular substituent moieties found on the compounds described herein. When formulations of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When formulations of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific formulations of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the formulations of the present invention. Additionally, prodrugs can be converted to the formulations of the present invention by chemical or biochemical methods in an ex vivo environment. For example, prodrugs can be slowly converted to the formulations of the present invention when placed in a transdermal patch reservoir with a suitable enzyme or chemical reagent.

Certain formulations of the present invention can exist in unsolvated forms as well as solvated forms, including hydrated forms. In general, the solvated forms are equivalent to unsolvated forms and are encompassed within the scope of the present invention. Certain formulations of the present invention may exist in multiple crystalline or amorphous forms. In general, all physical forms are equivalent for the uses contemplated by the present invention and are intended to be within the scope of the present invention.

Certain formulations of the present invention possess asymmetric carbon atoms (optical centers) or double bonds; the racemates, diastereomers, tautomers, geometric isomers and individual isomers are encompassed within the scope of the present invention. The formulations of the present invention do not include those which are known in the art to be too unstable to synthesize and/or isolate.

The formulations of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the formulations of the present invention, whether radioactive or not, are encompassed within the scope of the present invention.

The term “treating” refers to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the methods of the invention successfully treat a patient's delirium by decreasing the incidence of disturbances in consciousness or cognition.

As used herein, the term “cancer” refers to all types of cancer, neoplasm, or malignant tumors found in mammals, including leukemia, carcinomas and sarcomas. Exemplary cancers include cancer of the brain, breast, cervix, colon, head & neck, liver, kidney, lung, non-small cell lung, melanoma, mesothelioma, ovary, sarcoma, stomach, uterus and Medulloblastoma. Additional examples include, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P₃₈₈ leukemia model is widely accepted as being predictive of in vivo anti-leukemic activity. It is believed that a compound that tests positive in the P₃₈₈ assay will generally exhibit some level of anti-leukemic activity in vivo regardless of the type of leukemia being treated. Accordingly, the present invention includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas which can be treated include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chlioroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas which can be treated include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniformi carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lyinphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “antineoplastic” means inhibiting or preventing the growth of cancer. “Inhibiting or preventing the growth of cancer” includes reducing the growth of cancer relative to the absence of a given therapy or treatment. Cytotoxic assays useful for determining whether a compound is antineoplastic are well know in the art of cancer therapy and are available for a wide variety of cancers.

As used herein “combination therapy” or “adjunct therapy” means that the patient in need of the drug is treated or given another drug for the disease in conjunction with the formulations of the present invention. This combination therapy can be sequential therapy where the patient is treated first with one drug and then the other or the two drugs are given simultaneously. The present invention includes combination therapy or adjunct therapy using the water soluble formulations of the present invention.

“Patient” refers to a mammalian subject, including human.

As used herein, the term “wet age-related macular degeneration (AMD)” refers to an eye condition or disease in which damaging new blood vessel growth and leakage occurs in the retina, and if left untreated can lead to vision loss. AMD is the leading cause of age related blindness.

As used herein, the term “diabetic retinopathy” refers to an ocular pathology associated with diabetes. Diabetes can cause damage to the blood vessels that nourish the retina, and this can cause the vessels to leak or break, stimulating the growth of abnormal new blood vessels. Diabetic retinopathy is one of the leading causes of blindness in diabetics, and affects more than 4 million adults in America alone.

### II. Introduction

It has been discovered that non-ionic surfactants may be used to increase the solubility and/or bioavailability of prenylflavonoid or prenylflavonoid metabolites in water soluble formulations. Thus, the novel combination of a prenylflavonoid or prenylflavonoid metabolite and a non-ionic surfactant in a water soluble formulation provides an unexpected improvement in the administration of prenylflavonoids.

### III. Water Soluble Formulations

In one aspect, the present invention provides a water-soluble formulation including a prenylflavonoid or prenylflavonoid metabolite, and a non-ionic surfactant. A “prenylflavonoid,” as used herein, refers to a prenylated compound having a substituted or unsubstituted phenol attached to a phenyl via a C₃ alkylene substituted with an oxo group. The C₃ alkylene may be present in a linear chain arrangement (e.g. a chalcone) or joined with other atoms to form a substituted or unsubstituted ring (e.g. a flavanone). Prenylflavonoids may be derived from natural sources (e.g. hops), or synthesized chemically. Tabat et al., Phytochemistry 46: 683-687 (1997).

As used herein, a “prenylated” compound refers to those compounds with an attached —CH₂—CH═C(CH₃)₂ group (e.g. geranylated compounds), optionally hydroxylated prenyl tautomers (e.g. —CH₂—CH—C(CH₃)═CH₂, or —CH₂—C(OH)—C(CH₃)═CH₂), and optionally hydroxylated circularized prenyl derivatives having the formula:

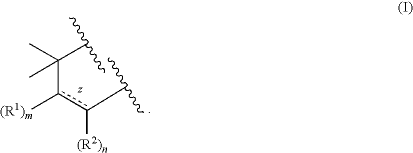

In Formula (I), the dashed bond z represents a double bond or a single bond. R¹ and R² are independently hydrogen or OH. The symbol [symbol] represents the point of attachment to the remainder of the prenylated compounds.

Thus, prenylflavonoids useful in the present invention include prenylchalcones and/or prenylflavanones. In some embodiments, the prenylflavonoid is selected from xanthohumol, xanthogalenol, desmethylxanthobumol (2′,4′,6′,4-tetrahydrooxy-3-C-prenylchalcone), 2′,4′,6′,4-tetrahydrooxy-3′-C-geranylchalcone, dehydrocycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,8-diprenylnaringenin, 4′,6′-dimethoxy-2′,4-dihydroxychalcone, 4′-O-methylxanthohumol, 6-geranylnaringenin, 8-geranylnaringenin, and metabolites and/or derivatives thereof. In some embodiments, the prenylflavonoid is xanthohumol, a xanthohumol metabolite, or derivative thereof. In some embodiments, the prenylflavonoid is xanthohumol.

The prenylflavonoid may derived from a natural source, such as hops. Thus, the water-soluble formulation may include hops or hops extract, and a non-ionic surfactant, wherein the hops or hops extract includes a prenylflavonoid. Prenylflavonoids may be isolated from hops through purification, fractionation, or separation methods that are known to those skilled in the art. See, for example, Tabata et. al., Phytochemistiy 46(4): 683-687 (1997).

A “non-ionic surfactant,” as used herein, is a surface active agent that tends to be non-ionized (i.e. uncharged) in neutral solutions (e.g. neutral aqueous solutions). Useful non-ionic surfactants include, for example, non-ionic water soluble mono-, di-, and tri-glycerides; non-ionic water soluble mono- and di-fatty acid esters of polyethyelene glycol; non-ionic water soluble sorbitan fatty acid esters (e.g. sorbitan monooleates such as SPAN 80 and TWEEN 20 (polyoxyethylene 20 sorbitan monooleate)); polyglycolyzed glycerides; non-ionic water soluble triblock copolymers (e.g. poly(ethyleneoxide)/poly-(propyleneoxide)/poly(ethyleneoxide) triblock copolymers such as POLOXAMER 406 (PLURONIC F-127), and derivatives thereof.

Examples of non-ionic water soluble mono-, di-, and tri-glycerides include propylene glycol dicarpylate/dicaprate (e.g. MIGLYOL 840), medium chain mono- and diglycerides (e.g. CAPMUL and IMWITOR 72), medium-chain triglycerides (e.g. caprylic and capric triglycerides such as LAVRAFAC, MIGLYOL 810 or 812, CRODAMOL GTCC-PN, and SOFTISON 378), long chain monoglycerides (e.g. glyceryl monooleates such as PECEOL, and glyceryl monolinoleates such as MAISINE), polyoxyl castor oil (e.g. macrogolglycerol ricinoleate, macrogolglycerol hydroxystearate, macrogol cetostearyl ether), and derivatives thereof.

Non-ionic water soluble mono- and di-fatty acid esters of polyethyelene glycol include d-α-tocopheryl polyethyleneglycol 1000 succinate (TPGS), poyethyleneglycol 660 12-hydroxystearate (SOLUTOL HS 15), polyoxyl oleate and stearate (e.g. PEG 400 monostearate and PEG 1750 monostearate), and derivatives thereof.

Polyglycolyzed glycerides include polyoxyethylated oleic glycerides, polyoxyethylated linoleic glycerides, polyoxyethylated caprylic/capric glycerides, and derivatives thereof. Specific examples include LABRAFIL M-1944CS, LABRAFILM-2125CS, LABRASOL, SOFTIGEN, and GELUCIRE.

In some embodiments, the non-ionic surfactant is a polyoxyl castor oil, or derivative thereof. Effective polyoxyl castor oils may be synthesized by reacting either castor oil or hydrogenated castor oil with varying amounts of ethylene oxide. Macrogolglycerol ricinoleate is a mixture of 83% relatively hydrophobic and 17% relatively hydrophilic components. The major component of the relatively hydrophobic portion is glycerol polyethylene glycol ricinoleate, and the major components of the relatively hydrophilic portion are polyethylene glycols and glycerol ethoxylates. Macrogolglycerol hydroxystearate is a mixture of approximately 75% relatively hydrophobic of which a major portion is glycerol polyethylene glycol 12-oxystearate.

In some embodiments, the water soluble formulation is a non-alcoholic formulation. A “non-alcoholic” formulation, as used herein, is a formulation that does not include (or includes only in trace amounts) methanol, ethanol, propanol or butanol. In other embodiments, the formulation does not include (or includes only in trace amounts) ethanol.

In some embodiments, the formulation is a non-aprotic solvated formulation. The term “non-aprotic solvated,” as used herein, means that water soluble aprotic solvents are absent or are included only in trace amounts. Water soluble aprotic solvents are water soluble non-surfactant solvents in which the hydrogen atoms are not bonded to an oxygen or nitrogen and therefore cannot donate a hydrogen bond.

In some embodiments, the water soluble formulation does not include (or includes only in trace amounts) a polar aprotic solvent. Polar aprotic solvents are aprotic solvents whose molecules exhibit a molecular dipole moment but whose hydrogen atoms are not bonded to an oxygen or nitrogen atom. Examples of polar aprotic solvents include aldehydes, ketones, dimethyl sulfoxide (DMSO), and dimethyl formamide (DMF). In other embodiments, the water soluble formulation does not include (or includes only in trace amounts) dimethyl sulfoxide. Thus, in some embodiments, the water soluble formulation does not include DMSO. In a related embodiment, the water soluble formulation does not include DMSO or ethanol.

In still other embodiments, the water soluble formulation does not include (or includes only in trace amounts) a non-polar aprotic solvent. Non-polar aprotic solvents are aprotic solvents whose molecules exhibit a molecular dipole of approximately zero. Examples include hydrocarbons, such as alkanes, alkenes, and alkynes.

The water soluble formulation of the present invention includes formulations dissolved in water (i.e. aqueous formulations).

In some embodiments, the water soluble formulation consists essentially of a prenylflavonoid or prenylflavonoid metabolite, a non-ionic surfactant. A “water soluble formulation consists essentially of a prenylflavonoid or prenylflavonoid metabolite, a non-ionic surfactant” means that the formulation includes a prenylflavonoid or prenylflavonoid metabolite, a non-ionic surfactant, and optionally additional components widely known in the art to be useful in neutraceutical formulations, such as preservatives, taste enhancers, buffers, water, etc. A “water soluble formulation consists essentially of a prenylflavonoid or prenylflavonoid metabolite, a non-ionic surfactant,” as used herein, does not include components that would destroy the novelty and inventiveness of the formulation.

### IV. Methods

In another aspect, the present invention provides a method of treating cancer, obesity, diabetes, cardiovascular disease, dyslipidaemia, age-related macular degeneration (e.g. vision loss associated with age-related macular degeneration), high cholesterol, or retinopathy (e.g. diabetic retinopathy) in subject in need of such treatment. The method includes administering to the subject an effective amount of the water soluble formulation of the present invention. The term “cancer” is defined in detail above.

In some embodiments, a method of lowering cholesterol in a subject in need of cholesterol lowering therapy is provided. The method includes administering to the subject an effective amount of the water soluble formulation of the present invention. The cholesterol lowering may be total cholesterol lowering or low density lipoprotein (LDL) lowering.

In another aspect, the present invention provides a method of treating a VEGF-mediated disease state in a subject in need of such treatment. The method includes administering to the subject an effective amount of the water soluble formulation of the present invention.

In some embodiments, a method is provided for reducing VEGF-mediated vascular permeability and/or abnormal blood vessel growth in the retina of a subject in need of such treatment. The method includes administering to the subject an effective amount of the water soluble formulation of the present invention.

In other embodiments, a method is provided for treating age-related macular degeneration in a subject in need of such treatment. The method includes administering to the subject an effective amount of the water soluble formulation of the present invention.

In still other embodiments, a method is provided for treating diabetic macular edema in a subject in need of such treatment. The method includes administering to the subject an effective amount of the water soluble formulation of the present invention.

Vascular endothelial growth factor (VEGF) is a diffusible protein that is specific to vascular endothelial cells and plays a major role in the regulation of physiological and pathological growth of blood vessels. VEGF promotes the growth of vascular endothelial cells that reside in arteries, veins, and lymphatics, but also has the ability to induce vascular leakage. This permeability enhancing activity is a connecting link between this molecule and other pathological states. For example, VEGF is expressed in the majority of human tumors and plays a critical role in tumor angiogenesis and metastasis. In addition, VEGF is directly involved in the pathological process that leads to the cancer, vision loss associated with age-related macular degeneration (including wet age-related macular degeneration), and retinopathies (such as diabetic retinopathy/diabetic macular edema).

Therefore, in some embodiments, a method of reducing the activity of VEGF is provided. The method may be conducted in vitro or in situ for research purposes by contacting VEGF with the water soluble formulation of the present invention. Alternatively, the activity of VEGF may be reduced in a subject by administering to the subject an effective amount of the water soluble formulation of the present invention.

VEGF inhibition can be measured in-vitro in a suitable cell line such as KOP2.16 endothelial cells, or using other techniques such as the Miles assay.

In another aspect, the present invention provides a method of treating a DGAT-mediated disease state in a subject in need of such treatment. The method includes administering to the subject an effective amount of the water soluble formulation of the present invention.

Acyl CoA:diacylglycerol acyltransferase (DGAT) is a ubiquitously expressed microsomal enzyme that catalyzes the final reaction in the major pathways of triglyceride synthesis. Mice deficient in the DGAT enzyme are resistant to diet induced obesity and have increased insulin and leptin sensitivity. Research suggests that therapeutic inhibition of DGAT in-vivo results in effective treatment of both obesity and diabetes. Thus, in some embodiments, the DGAT-mediated disease state is obesity, diabetes, cardiovascular disease, and/or dyslipidaemia (including elevated cholesterol, elevated triglycerides, and/or dyslipidaemia associated with diabetes). The water soluble formulations of the present invention may also be employed to increase the metabolic rate or energy level of a subject.

Therefore, in some embodiments, a method of reducing the activity of DGAT is provided. The method may be conducted in vitro or in situ for research purposes by contacting DGAT with the water soluble formulation of the present invention. Alternatively, the activity of DGAT may be reduced in a subject by administering to the subject an effective amount of the water soluble formulation of the present invention.

In another aspect, the present invention provides a method of treating an ACAT-mediated disease state in a subject in need of such treatment. The method includes administering to the subject an effective amount of the water soluble formulation of the present invention. In some embodiments, the disease state is obesity, diabetes, cardiovascular disease, and/or dyslipidaemia (including elevated cholesterol, elevated triglycerides, and/or dyslipidaemia associated with diabetes).

Acyl-coenzyme A cholesterol acyl transferase (ACAT) is an enzyme that esterifies cholesterol. For unesterified “free” cholesterol to be packaged into ApoB-containing lipoproteins in the liver, it must be esterified by ACAT. ACAT inhibition is believed to be antiatherogenic by accelerating cholesterol excretion by the liver, as well as by inhibiting cholesterol absorption in the intestines. ACAT inhibition also may prevent cholesteryl ester accumulation in macrophages in the arterial walls, which results in antiatherosclerosis effects. ACAT inhibition may have direct effects on the vascular system through impairment of conversion of free cholesterol to esterified cholesterol in endothelial macrophage by reducing foam cell formation. Normally, ACAT inhibitors are thought to prevent accumulation of lipid in the arterial wall without significantly affecting plasma lipid levels.

In some embodiments, a method of reducing the activity of ACAT is provided. The method may be conducted in vitro or in situ for research purposes by contacting ACAT with the water soluble formulation of the present invention. Alternatively, the activity of ACAT may be reduced in a subject by administering to the subject an effective amount of the water soluble formulation of the present invention.

In another aspect, the present invention provides a method for enhancing the bioavailability of a prenylflavonoid or prenylflavonoid metabolite in a subject. The method includes combining said prenylflavonoid or prenylflavonoid metabolite, and a non-ionic surfactant to form a surfactant-prenylflavonoid mixture. The surfactant-prenylflavonoid mixture is administered to the subject thereby enhancing the bioavailability of the prenylflavonoid or prenylflavonoid metabolite. The bioavailability is enhanced compared to the bioavailability of the prenylflavonoid in the absence of non-ionic surfactant.

In another aspect, the present invention provides a method of dissolving a prenylflavonoid in water. The method includes combining a prenylflavonoid with a non-ionic surfactant to form a surfactant-prenylflavonoid mixture. The surfactant-prenylflavonoid mixture is combined with water thereby dissolving the prenylflavonoid in water. The solution may be optionally heated to increase solubility. The heating temperature is typically selected to avoid chemical breakdown of the prenylflavanoid and/or non-ionic surfactant.

A subject is an organism that is treated using one of the methods of the present invention. In some embodiment, the subject is a mammalian subject, such as a human or domestic animal.

An effective amount of the water soluble formulation of the present invention is an amount sufficient to achieve the intended purpose of a method of the present invention, such as treating a particular disease state in a subject (e.g. a human subject).

### V. Dosages and Dosage Forms

The amount of prenylflavonoid adequate to treat a disease (e.g. through modulation of DGAT, VEGF, and/or ACAT) is defined as a “therapeutically effective dose”. The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, prenylflavonoid and disease or condition treated.

Single or multiple administrations of prenylflavonoid formulations can be administered depending on the dosage and frequency as required and tolerated by the patient. The formulations should provide a sufficient quantity of active agent to effectively treat the disease state. Lower dosages can be used, particularly when the drug is administered to an anatomically secluded site in contrast to administration orally, into the blood stream, into a body cavity or into a lumen of an organ. Substantially higher dosages can be used in topical administration. Actual methods for preparing parenterally administrable prenylflavonoid formulations will be known or apparent to those skilled in the art and are described in more detail in such publications as Remington's, supra. See also Nieman, In “Receptor Mediated Antisteroid Action,” Agarwal, et al., eds., De Gruyter, N.Y. (1987).

In some embodiments, the prenylflavanoid is present in the water soluble formulation at a concentration of at least 5%, 10%, 20%, 25%, 30%, 35%, 45%, 45%, or 50% by weight. In other embodiments the prenylflavonoid is present in the water soluble formulation at a concentration from 0.01%, 0.1%, 1% to 80%, 5% to 50%, 10% to 35%, or 20% to 25% (by weight). The prenylflavonoid may also be present (e.g. in a beverage formulation) at a concentration from 0.5 to 5 mg per 4 fluid ounces, or around 1 mg per 4 fluid ounces. In other embodiments, the prenylflavonoid is present at a concentration from 0.01 mg/ml to 25 mg/ml. In some concentrated formulations (e.g. a soft gel tablet formulation), the prenylflavonoid may be present at about 1 to 5 mg/ml, or around 2 mg/ml, or at least 1 mg/ml.

In other embodiments, at least 0.5 mg, 1 mg, 2 mg, 3 mg, 4 mg, 5 mg, 10 mg, 20 mg, 30 mg, 40 mg, 50 mg, 100 mg, 200 mg, 300 mg, 400 mg, 500 mg, or 1 g of prenylflavonoid is present in the water soluble formulation. In other embodiments, 0.1 mg to 2 g, 0.5 mg to 1 g, 1 mg to 500 mg, 1 mg to 100 mg, 1 mg to 50 mg, 1 mg to 10 mg, or 1 mg to 5 mg of prenylflavonoid is present in the water soluble formulation.

In some embodiments, the water soluble formulation is in the form of a pharmaceutical composition. The pharmaceutical composition may include a prenylflavonoid, or prenylflavonoid metabolite, a non-ionic surfactant, and a pharmaceutically acceptable excipient. After a pharmaceutical composition including a prenylflavonoid of the invention has been formulated in an acceptable carrier, it can be placed in an appropriate container and labeled for treatment of an indicated condition. For administration of prenylflavonoids, such labeling would include, e.g., instructions concerning the amount, frequency and method of administration. In one embodiment, the invention provides for a kit for the treatment of delirium in a human which includes a prenylflavonoid and instructional material teaching the indications, dosage and schedule of administration of the prenylflavonoid.

Any appropriate dosage form is useful for administration of the water soluble formulation of the present invention, such as oral, parenteral and topical dosage forms. Oral preparations include tablets, pills, powder, dragees, capsules (e.g. soft-gel capsules), liquids, lozenges, gels, syrups, slurries, beverages, suspensions, etc., suitable for ingestion by the patient. The formulations of the present invention can also be administered by injection, that is, intravenously, intramuscularly, intracutaneously, subcutaneously, intraduodenally, or intraperitoneally. Also, the formulations described herein can be administered by inhalation, for example, intranasally. Additionally, the formulations of the present invention can be administered transdermally. The formulations can also be administered by in intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Thus, the formulations described herein may be adapted for oral administration.

For preparing pharmaceutical compositions from the formulations of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa. (“Remington's”).

Suitable carriers include magnesium carbonate, magnesium stearate, talc, sugar, lactose, pectin, dextrin, starch (from corn, wheat, rice, potato, or other plants), gelatin, tragacanth, a low melting wax, cocoa butter, sucrose, mannitol, sorbitol, cellulose (such as methyl cellulose, hydroxypropylmethyl-cellulose, or sodium carboxymethylcellulose), and gums (including arabic and tragacanth), as well as proteins such as gelatin and collagen. If desired, disintegrating or co-solubilizing agents may be added, such as the cross-linked polyvinyl pyrrolidone, agar, alginic acid, or a salt thereof, such as sodium alginate. In powders, the carrier is a finely divided solid, which is in a mixture with the finely divided active component. In tablets, the active component is mixed with the carrier having the necessary binding properties in suitable proportions and compacted in the shape and size desired.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain prenylflavonoid mixed with a filler or binders such as lactose or starches, lubricants such as talc or magnesium stearate, and, optionally, stabilizers. In soft capsules, the prenylflavonoid compounds may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, beverages, and emulsions, for example, water or water/propylene glycol solutions. For parenteral injection, liquid preparations can be formulated in solution in aqueous polyethylene glycol solution.

Aqueous solutions and beverages suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending a prenylflavonoid in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The formulations of the invention can be delivered transdermally, by a topical route, formulated as applicator sticks, solutions, suspensions, emulsions, gels, creams, ointments, pastes, jellies, paints, powders, and aerosols.

The formulations can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The formulations of the invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use

In another embodiment, the formulations of the invention are useful for parenteral administration, such as intravenous (IV) administration or administration into a body cavity or lumen of an organ. The formulations for administration will commonly comprise a solution of the prenylflavonoid dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of prenylflavonoid in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the prenylflavonoid into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The formulations may be administered as a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the active component. The composition can, if desired, also contain other compatible therapeutic agents.

### VI. Assays

Subject non-ionic surfactants may be assayed for their ability to solubilize a prenylflavonoid or prenylflavonoid metabolite using any appropriate method. Typically, a non-ionic surfactant is contacted with the prenylflavonoid and mixed mechanically and/or automatically using a shaker or sonicator device. Water may be optionally added, for example, where the prenylflavonoid and/or surfactant is in powder form. The solution may be optionally heated to increase solubility. The heating temperature is selected to avoid chemical breakdown of the prenylflavanoid and non-ionic surfactant.

The resulting solution may be visually inspected for colloidal particles to determine the degree of solubility of the prenylflavonoid. Alternatively, the solution may be filtered and analyzed to determine the degree of solubility. For example, a spectrophotometer may be used to determine the concentration of prenylflavonoid present in the filtered solution. Typically, the test solution is compared to a positive control containing a series of known quantities of pre-filtered prenylflavonoid solutions to obtain a standard concentration versus UV/vis absorbance curve. Alternatively, high performance liquid chromatography may be used to determine the amount of prenylflavonoid in solution.

High throughput solubility assay methods are well known in the art. Typically, these methods involve automated dispensing and mixing of solutions with varying amounts of non-ionic surfactants, prenylflavonoid, and optionally other co-solvents. The resulting solutions may then be analyzed to determine the degree of solubility using any appropriate method as discussed above.

For example, the Millipore MultiScreen Solubility filter Plate® with modified track-etched polycarbonate, 0.4 μm membrane is a single-use, 96-well product assembly that includes a filter plate and a cover. The device is intended for processing aqueous solubility samples in the 100-300 μL volume range. The vacuum filtration design is compatible with standard, microtiter plate vacuum manifolds. The plate is also designed to fit with a standard, 96-well microtiter receiver plate for use in filtrate collection. The MultiScreen Solubility filter Plate® has been developed and QC tested for consistent filtration flow-time (using standard vacuum), low aqueous extractable compounds, high sample filtrate recovery, and its ability to incubate samples as required to perform solubility assays. The low-binding membrane has been specifically developed for high recovery of dissolved organic compounds in aqueous media.

The aqueous solubility assay allows for the determination of prenylflavonoid solubility by mixing, incubating and filtering a solution in the MultiScreen Solubility filter plate. After the filtrate is transferred into a 96-well collection plate using vacuum filtration, it is analyzed by UV/Vis spectroscopy to determine solubility. Additionally, LC/MS or HPLC can be used to determine compound solubility, especially for compounds with low UV/Vis absorbance and/or compounds with lower purity. For quantification of aqueous solubility, a standard calibration curve may be determined and analyzed for each compound prior to determining aqueous solubility.

Test solutions may be prepared by adding an aliquot of concentrated drug or compound. The solutions are mixed in a covered 96-well MultiScreen Solubility filter plate for 1.5 hours at room temperature. The solutions are then vacuum filtered into a 96-well, polypropylene, V-bottomed collection plate to remove any insoluble precipitates. Upon complete filtration, 160 μL/well are transferred from the collection plate to a 96-well UV analysis plate and diluted with 40 μL/well of acetonitrile. The UV/vis analysis plate is scanned from 260-500 nm with a UV/vis microplate spectrometer to determine the absorbance profile of the test compound.

Thus, one skilled in the art may assay a wide variety of non-ionic surfactants to determine their ability of solubilize various prenylflavonoid compounds.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, the features of the formulations are equally applicable to the methods of treating disease states described herein. All publications, patents, and patent applications cited herein are hereby incorporated by reference in their entirety for all purposes.

### VII. Examples

The examples below are meant to illustrate certain embodiments of the invention, and are intended to limit the scope of the invention.

Lucifer Yellow was purchased from Molecular Probes (Eugene, Oreg.). Hanks buffer and all other chemicals were obtained from Sigma-Aldrich (St. Louis, Mo.).

### Example 1

Water soluble compositions of xanthohumol were formulated containing the non-ionic surfactant macrogolglycerol hydroxystearate. By heating and stirring this polyoxyl castor oil with a powdered xanthohumol extract (containing in excess of 20% xanthohumol by weight), a clear greenish viscous solution was formed containing dissolved xanthohumol (hereinafter referred to as “xanthohumol gel formulation”) a clear greenish viscous solution was formed containing dissolved xanthohumol. The powdered xanthohumol extract consisted of 20% xanthohumol, small amounts of chlorophyll, and uncharacterized residual resins, but did not contain any alpha acids, beta acids, or 8-prenylnaringenin. The xanthohumol gel formulation consisted of macrogolglycerol hydroxystearate 40 (100 ml) and powdered xanthohumol extract (10 grams), representing a ratio of surfactant:prenylflavonoid of 10:1.

An aqueous solution of solubilized xanthohumol was achieved by adding water to the xanthohumol gel formulation (hereinafter referred to as “aqueous xanthohumol formulation”). More specifically, the aqueous xanthohumol formulation was prepared by warming the xanthohumol gel formulation in warm water to form a clear aqueous solution of xanthohumol. This aqueous xanthohumol formulation did not have undesirable flavor. The aqueous xanthohumol formulation consisted of water (200 ml), macrogolglycerol hydroxystearate 40 (100 ml), and powdered xanthohumol extract (10 grams), representing a ratio of 20:10:1 for the water:surfactant:prenylflavonoid. The aqueous xanthohumol formulation was analyzed by HPLC and found to contain 0.6%, or 6 mg/ml xanthohumol.

### Example 2

HMG-CoA reductase assays were performed in which increasing concentrations of xanthohumol (1 μM to 100 μM) were added to isolated liver microsomes. Xanthohumol had no effect on HMG-CoA reductase activity. As a positive control, atorvastatin (10 nM and 1 μM) was tested in the same assay, which inhibited reductase activity by 58% and 87% respectively. The protocol followed was as published in Telford et al. ATVB 2002; 22: 1884-1891.

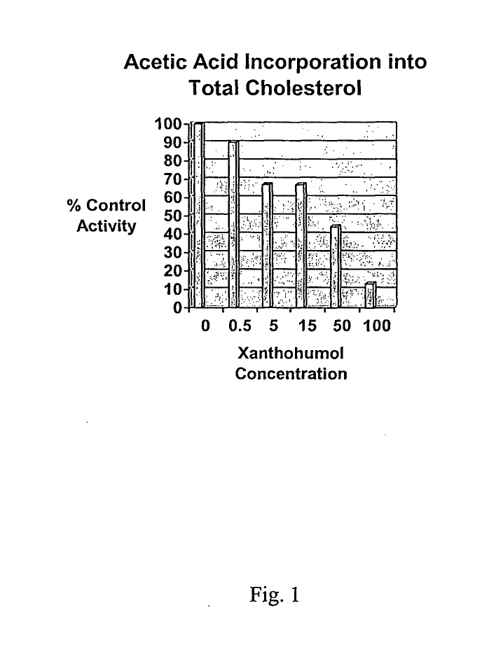

The incorporation of ¹⁴C-acetic acid into cholesterol was examined in HepG2 cells. No affect was observed for this parameter for concentrations of xanthohumol below 500 nM. Above this concentration, cholesterol synthesis was inhibited in a dose-responsive manner (0.5 μM to 100 μM). See FIG. 1. The IC₅₀ was approximately 20 μM. In the same HepG2 cell assay system, atorvastatin (10 nM and 1 μM) inhibited acetate incorporation into cholesterol by 20% and 80% respectively.

### Example 3

The solubility of the powdered xanthohumol extract in pH 7.4 Hank's Balanced Salt Solution (10 mM HEPES and 15 mM glucose) was compared to the xanthohumol gel formulation. At least 1 mg of powdered xanthohumol extract or 100 mg of xanthohumol gel formulation was combined with 1 ml of buffer to make a ≧1 mg/ml powdered xanthohumol extract mixture and a ≧1 mg/ml xanthohumol gel formulation mixture, respectively. The mixtures were shaken for 2 hours using a benchtop vortexer and left to stand overnight at room temperature. After vortexing and standing overnight, the powdered xanthohumol extract mixture was then filtered through a 0.45-μm nylon syringe filter (Whatman, Cat# 6789-0404) that was first saturated with the sample.

After vortexing and standing overnight, the xanthohumol gel formulation mixture was centrifuged at 14,000 rpm for 10 minutes. The filtrate or supernatant was sampled twice, consecutively, and diluted 10, 100, and 10,000-fold in a mixture of 50:50 assay buffer:acetonitrile prior to analysis.

Both mixtures were assayed by LC/MS/MS using electrospray ionization against the standards prepared in a mixture of 50:50 assay buffer:acetonitrile. Standard concentrations ranged from 1.0 μM down to 3.0 nM. Results are presented in Table 1 below.

TABLE 1
Solubility of Xanthohumol in pH 7.4 Phosphate Buffer

| | Solubility (μM) | | |
|---|---|---|---|
| Test Article Identification | Rep 1 | Rep 2 | AVG |
| Powdered Xanthohumol | 0.40 | 0.81 | 0.61 |
| Extract | | | |
| Xanthohumol Gel | 1860 | 1700 | 1780 |
| Formulation | | | |

As shown in Table 1, the powdered xanthohumol extract and xanthohumol gel formulation gel showed average solubility values in pH 7.4 Hank's Balanced Salt Solution of 0.61 μM and 1780 μM, respectively.

### Example 4

The permeability of the xanthohumol gel through a cell-free (blank) microporous 0.4 micron membrane filter was studied in order to determine the non-specific binding and cell-free diffusion Pₐₚₚ of the xanthohumol gel formulation through the filter. The xanthohumol gel formulation was assayed at the 2 μM xanthohumol concentration in Hanks buffer (Hanks Balanced Salt Solution (HBSSg) containing 10 mM HEPES and 15 mM glucose) at a pH of 7.4 in duplicate. Donor samples were collected at 120 minutes. Receiver samples were collected at 60 and 120 minutes. The apparent permeability coefficient, Pₐₚₚ, and percent recovery were calculated as follows:

Pₐₚₚ=(dCᵣ/dt)×Vᵣ/(A×C₀)

Percent Recovery=100×((Vᵣ×Cᵣ^{final})+(V_{d}×C_{d}^{final}))/(V_{d}×C₀)
Where:
dCᵣ/dt is the slope of the cumulative concentration in the receiver compartment versus time in μM s⁻¹.
Vᵣ is the volume of the receiver compartment in cm³.
V_{d} is the volume of the donor compartment in cm³.
A is the area of the cell-free insert (1.13 cm² for 12-well Transwell).
Cᵣ^{final} is the cumulative receiver concentration in μM at the end of the incubation period.
C_{d}^{final} is the concentration of the donor in μM at the end of the incubation period.
C₀ is the initial concentration of the dosing solution in μM.

Results of the non-specific binding assessment are presented in Table 2, which shows the permeability (10⁻⁶ cm/s) and recovery of Xanthohumol across the cell-free filter.

TABLE 2

| Xanthohumol Dosing | | |
|---|---|---|
| Solution Concentration (μM) | Pₐₚₚ (10⁻⁶ cm/s) | |
| (Average, N = 2) | A-to-B^{A} | Recovery (%)^{B} |
| Rep. 1: 2.31 | Rep. 1: 18.6 | Rep. 1: 95 |
| Rep. 2: 2.46 | Rep. 2: 17.1 | Rep. 2: 99 |
| AVERAGE: 2.39 | AVERAGE: 17.9 | AVERAGE: 97 |

^{A}A low rate of diffusion (<20 × 10⁻⁶ cm/s) through the cell-free membrane may indicate a lack of free diffusion, which may affect the measured permeability.
^{B}Low recoveries caused by non-specific binding, etc. would affect the measured permeability.

### Example 5

To test the permeability of xanthohumol across Caco-2 cell monolayers, Caco-2 cell monolayers were grown to confluence on collagen-coated, microporous, polycarbonate membranes in 12-well Costar Transwell® plates. Details of the plates and their certification are shown below in Table 3. The test article was also the aqueous xanthohumol formulation, and the dosing concentration was 2 μM in the assay buffer (HBSSg) as in the previous example. Cell monolayers were dosed on the apical side (A-to-B) or basolateral side (B-to-A) and incubated at 37° C. with 5% CO₂ in a humidified incubator. Samples were taken from the donor chamber at 120 minutes, and samples from the receiver chamber were collected at 60 and 120 minutes. Each determination was performed in duplicate. Lucifer yellow permeability was also measured for each monolayer after being subjected to the test article to ensure no damage was inflicted to the cell monolayers during the permeability experiment. All samples were assayed for Xanthohumol by LC/MS/MS using electrospray ionization. The apparent permeability (Pₐₚₚ), and percent recovery were calculated as described above. Xanthohumol permeability results are presented in Table 4, which shows the permeability (10⁻⁶ cm/s) and recovery of Xanthohumol across Caco-2 cell monolayers. All monolayers passed the post-experiment integrity control with Lucifer yellow Papp<0.8×10-6 cm/s.

TABLE 3

| Plates | TW12 |
|---|---|
| Seed Date | Jun. 6, 2006 |
| Passage Number | 63 |
| Age (Days) | 22 |

| Parameter | Value | Acceptance Criteria |
|---|---|---|
| TEER Value (Ω · cm²) | 468 | 450-650 |
| Lucifer Yellow Pₐₚₚ, × 10⁻⁶ cm/s | 0.13 | <0.4 |
| Atenolol Pₐₚₚ, × 10⁻⁶ cm/s | 0.30 | <0.5 |
| Propranolol Pₐₚₚ, × 10⁻⁶ cm/s | 20.65 | 15-25 |
| Digoxin (B-to-A)/(A-to-B) Pₐₚₚ Ratio | 16.57 | >3 |

TABLE 4

| | | Dosing Conc. | Percent | Pₐₚₚ | Efflux | Significant | Absorption |
|---|---|---|---|---|---|---|---|
| Test Article | Direction | (μM) | Recovery^{C} | (10⁻⁶ cm/s) | Ratio | Efflux^{B} | Potential^{A} |
| Xanthohumol | A-to-B | Rep. 1: 2.07 | Rep. 1: 30 | Rep. 1: 0.94 | 2.1 | No | Medium |
| | | Rep. 2: 2.03 | Rep. 2: 28 | Rep. 2: 0.74 | | | |
| | | Average 2.05 | Average: 29 | Average: 0.84 | | | |
| | B-to-A | Rep. 1: 2.25 | Rep. 1: 81 | Rep. 1: 1.36 | | | |
| | | Rep. 2: 2.21 | Rep. 2: 80 | Rep. 2: 2.18 | | | |
| | | Average: 2.23 | Average: 81 | Average: 1.77 | | | |

^{A}Absorption Potential Classification:
Pₐₚₚ(A-to-B) ≧ 1.0 × 10⁻⁶ cm/s High
1.0 × 10⁻⁶ cm/s > Pₐₚₚ(A-to-B) ≧ 0.5 × 10⁻⁶ cm/s Medium
Pₐₚₚ(A-to-B) < 0.5 × 10⁻⁶ cm/s Low
^{B}Efflux considered significant if:
Pₐₚₚ(B-to-A) ≧ 1.0 × 10⁻⁶ cm/s and Ratio Pₐₚₚ(B-to-A)/Pₐₚₚ(A-to-B) ≧ 3.0
^{C}Low recoveries caused by non-specific binding, etc. can affect the measured permeability.

### Example 6

The following formulation was prepared as described below: purified xanthohumol 98% (5% by weight), propylene glycol (15% by weight), Flavor (q.s.), povidone (10% by weight), and water (70% by weight).

Propylene glycol was warmed to about 100° F., and the purified xanthohumol (98%) was mixed until a clear yellowish solution was obtained. The warm mixture was slowly added to the water while mixing. Finally, the povidone and flavor was added.

### Example 7

The following formulation was prepared as described below: 8-prenylnaringenin 98% (10% by weight), macrogolglycerol hydroxystearate 40 (90% by weight).

The macrogolglycerol hydroxystearate 40 was warmed until clear. The 8-prenylnaringenin was slowly mixed or vortexed into solution until invisible. The resulting solution was clear. This clear solution is optionally added to water and flavored to create a pleasant tasting beverage, or encapsulated into a soft gel capsule.

What is claimed is:

1. A water-soluble formulation comprising:
a) a prenylflavonoid or prenylflavonoid metabolite; and
b) a non-ionic surfactant.

2. The formulation of claim 1, wherein said prenylflavonoid is a prenylchalcone or a prenylflavanone.

3. The formulation of claim 1, wherein said prenylflavonoid is selected from the group consisting of xanthohumol, xanthogalenol, desmethylxanthohumol (2′,4′,6′,4-tetrahydrooxy-3-C-prenylchalcone), 2′,4′,6′,4-tetrahydrooxy-3′-C-geranylchalcone, dehydrocycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,8-diprenylnaringenin, 4′,6′-dimethoxy-2′,4-dihydroxychalcone, 4′-O-methylxanthohumol, 6-geranylnaringenin, and 8-geranylnaringenin.

4. The formulation of claim 1, consisting essentially of:
a) a prenylflavonoid or prenylflavonoid metabolite; and
b) a non-ionic surfactant.

5. The formulation of claim 1, wherein said formulation is a non-alcoholic formulation.

6. The formulation of claim 1, wherein said formulation is a non-aprotic solvated formulation.

7. The formulation of claim 1, wherein said prenylflavonoid is present at a concentration of at least 0.01 mg/ml.

8. The formulation of claim 1, wherein said prenylflavonoid is present at a concentration of at least 1 mg/ml.

9. The formulation of claim 1, wherein said is present at a concentration of at least 0.01% by weight.

10. The formulation of claim 1, wherein said prenylflavonoid is present at a concentration of at least 20% by weight.

11. The formulation of claim 1, comprising from 1 mg to 5 mg of prenylflavonoid.

12. The formulation of claim 1, comprising at least 10 mg of prenylflavonoid.

13. The formulation of claim 1, wherein said non-ionic surfactant is a non-ionic water soluble mono-, di-, or tri-glyceride; non-ionic water soluble mono- or di-fatty acid ester of polyethyelene glycol; non-ionic water soluble sorbitan fatty acid ester; polyglycolyzed glyceride; non-ionic water soluble triblock copolymers; or derivative thereof.

14. The formulation of claim 1, wherein said non-ionic surfactant is a non-ionic water soluble mono-, di-, or tri-glyceride.

15. The formulation of claim 1, wherein said non-ionic surfactant is polyoxyl castor oil.

16. The formulation of claim 1, wherein said non-ionic surfactant is macrogolglycerol ricinoleate or macrogolglycerol hydroxystearate.

17. The formulation of claim 1, wherein said non-ionic surfactant is macrogolglycerol hydroxystearate.

18. The formulation of claim 1, wherein said formulation is an oral formulation.

19. The formulation of claim 18, wherein said oral formulation is a soft gel capsule.

20. The formulation of claim 18, wherein said oral formulation is a tablet.

21. The formulation of claim 18, wherein said oral formulation is a beverage.

22. The formulation of claim 1, wherein said formulation is an injectable formulation.

23. The formulation of claim 1, wherein said formulation is a topical formulation.

24. The formulation of claim 1, wherein said prenylflavonoid is derived from hops.

25. The formulation of claim 1, further comprising a pharmaceutically acceptable excipient.

26. The formulation of claim 1, wherein said prenylflavonoid is xanthohumol.

27. A method of dissolving a prenylflavonoid in water, said method comprising the steps of:
a. combining a prenylflavonoid with a non-ionic surfactant to form a surfactant-prenylflavonoid mixture; and
b. combining the surfactant-prenylflavonoid mixture with water thereby dissolving the prenylflavonoid in water.

28. The method of claim 27, wherein said prenylflavonoid is xanthohumol.

29. The method of claim 27, wherein said non-ionic surfactant is a polyoxyl castor oil.

30. A method of treating cancer, obesity, diabetes, cardiovascular disease, dyslipidaemia, vision loss associated with age-related macular degeneration, high cholesterol, or diabetic retinopathy in a subject in need of such treatment, said method comprising administering to the subject an effective amount of the formulation of claim 1.

31. A method of treating a VEGF-mediated disease state in a subject in need of such treatment, said method comprising administering to the subject an effective amount of the formulation of claim 1.

32. The method of claim 31, wherein said disease state is vision loss associated with age-related macular degeneration, or diabetic retinopathy.

33. A method of treating an ACAT-mediated disease state in a subject in need of such treatment, said method comprising administering to the subject an effective amount of the formulation of claim 1.

34. The method of claim 33, wherein said disease state is obesity, diabetes, cardiovascular disease, or dyslipidaemia.

35. A method of treating a DGAT-mediated disease state in a subject in need of such treatment, said method comprising administering to the subject an effective amount of the formulation of claim 1.

36. The method of claim 35, wherein said disease state is obesity, diabetes, cardiovascular disease, or dyslipidaemia.

37. A method of enhancing the bioavailability of a prenylflavonoid or prenylflavonoid metabolite in a subject, said method comprising the steps of:
(a) combining said prenylflavonoid or prenylflavonoid metabolite, and a non-ionic surfactant to form a surfactant-prenylflavonoid mixture; and
(b) administering said surfactant-prenylflavonoid mixture to said subject thereby enhancing the bioavailability of said prenylflavonoid or prenylflavonoid metabolite.
